(19) United States
(12) Patent Application Publication
(10) Pub. No.: US 2015/0196782 A1
(43) Pub. Date: Jul. 16, 2015

(54) ULTRASONIC PROBE AND ULTRASONIC TREATMENT DEVICE

(71) Applicant: OLYMPUS MEDICAL SYTEMS CORP., Tokyo (JP)

(72) Inventor: Tsunetaka AKAGANE, Hachioji-shi (JP)

(73) Assignee: OLYMPUS MEDICAL SYSTEMS CORP., Tokyo (JP)

(21) Appl. No.: 14/259,704

(22) Filed: Apr. 23, 2014

Related U.S. Application Data
(60) Provisional application No. 61/826,793, filed on May 23, 2013.

(51) Int. Cl. A61N 7/02; A61B 17/32

(52) U.S. Cl. CPC A61N 7/02; A61B 17/320092; A61B 2018/00589

(57) ABSTRACT

An ultrasonic probe is provided with a first vibrating body including a first distal treatment section which vibrates at a predetermined frequency and with a first amplitude when the ultrasonic vibration is transmitted. The ultrasonic probe includes a second vibrating body extending in a same range as the first vibrating body in axially parallel directions parallel to a longitudinal axis and being discontinuous with the first vibrating body over an entire length in the axially parallel directions. The second vibrating body includes a second distal treatment section vibrating at the same predetermined frequency as the first distal treatment section and with a second amplitude greater than the first amplitude when the ultrasonic vibration is transmitted.

### CROSS-REFERENCE TO RELATED APPLICATIONS

This application is based upon and claims the benefit of priority from prior U.S. Provisional Application No. 61/826,793, filed May 23, 2013, the entire contents of which are incorporated herein by reference.

### BACKGROUND OF THE INVENTION

1. Field of the Invention

The present invention relates to an ultrasonic probe configured to transmit an ultrasonic vibration from a proximal direction to a distal direction, and an ultrasonic treatment device including the ultrasonic probe.

2. Description of the Related Art

Japanese Patent No. 4700715 has disclosed an ultrasonic treatment device including an ultrasonic probe configured to transmit ultrasonic vibration from a proximal direction toward a distal direction. In this ultrasonic treatment device, a distal treatment section is provided to a distal portion of the ultrasonic probe. The ultrasonic treatment device also includes a jaw openable and closable relative to the distal treatment section. A treatment target is treated while being grasped between the distal treatment section of the ultrasonic probe and the jaw.

In the above-described ultrasonic treatment device configured to grasp the treatment target between the distal treatment section of the ultrasonic probe and the jaw, the distal treatment section includes an abutment surface which faces the jaw and on which the jaw can abut when the jaw is closed relative to the distal treatment section. A frictional heat is generated between the treatment target and the abutment surface when the ultrasonic probe is vibrated by the ultrasonic vibration while the treatment target is being grasped. The treatment target is cut by the frictional heat. Therefore, the abutment surface of the distal treatment section is used in the cutting treatment of the treatment target. In the ultrasonic treatment device configured to grasp the treatment target between the distal treatment section of the ultrasonic probe and the jaw, the distal treatment section also includes a noncontact surface which faces the jaw and which has a gap between this noncontact surface and the jaw while the jaw is in abutment with the abutment surface. If a high-frequency current is passed through the treatment target between the noncontact surface and the jaw while the treatment target is being grasped, the treatment target is denatured. As a result, the treatment target is coagulated. Therefore, the noncontact surface of the distal treatment section is used in the coagulation treatment of the treatment target.

### BRIEF SUMMARY OF THE INVENTION

According to one aspect of the invention, an ultrasonic probe configured to transmit an ultrasonic vibration from a proximal direction toward a distal direction along a longitudinal axis, the ultrasonic probe including: a first vibrating body which extends along the longitudinal axis, the first vibrating body including, at a distal portion thereof, a first distal treatment section which is configured to vibrate at a predetermined frequency and with a first amplitude when the ultrasonic vibration is transmitted; and a second vibrating body which extends along the longitudinal axis in a same range as the first vibrating body in axially parallel directions parallel to the longitudinal axis and which is discontinuous with the first vibrating body over an entire length in the axially parallel directions, the second vibrating body including, at a distal portion thereof, a second distal treatment section which is configured to vibrate at the same predetermined frequency as the first distal treatment section and with a second amplitude greater than the first amplitude when the ultrasonic vibration is transmitted.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing a configuration of an ultrasonic treatment device according to a first embodiment of the present invention;

FIG. 2 is a schematic sectional view showing an internal configuration of a vibrator case according to the first embodiment;

FIG. 3 is a schematic perspective view showing a configuration of an ultrasonic probe according to the first embodiment;

FIG. 4 is a sectional view taken along the line IV-IV in FIG. 3;

FIG. 5 is a schematic diagram showing a configuration of a distal portion of a handpiece according to the first embodiment;

FIG. 6 is a schematic sectional view showing a jaw and a distal portion of the ultrasonic probe according to the first embodiment in a section perpendicular to a longitudinal axis;

FIG. 7 is a schematic sectional view showing the distal portion of the ultrasonic probe according to a first modification in a section perpendicular to the longitudinal axis; and

FIG. 8 is a schematic diagram showing the configuration of an ultrasonic treatment device according to a second modification.

### DETAILED DESCRIPTION OF THE INVENTION

### First Embodiment

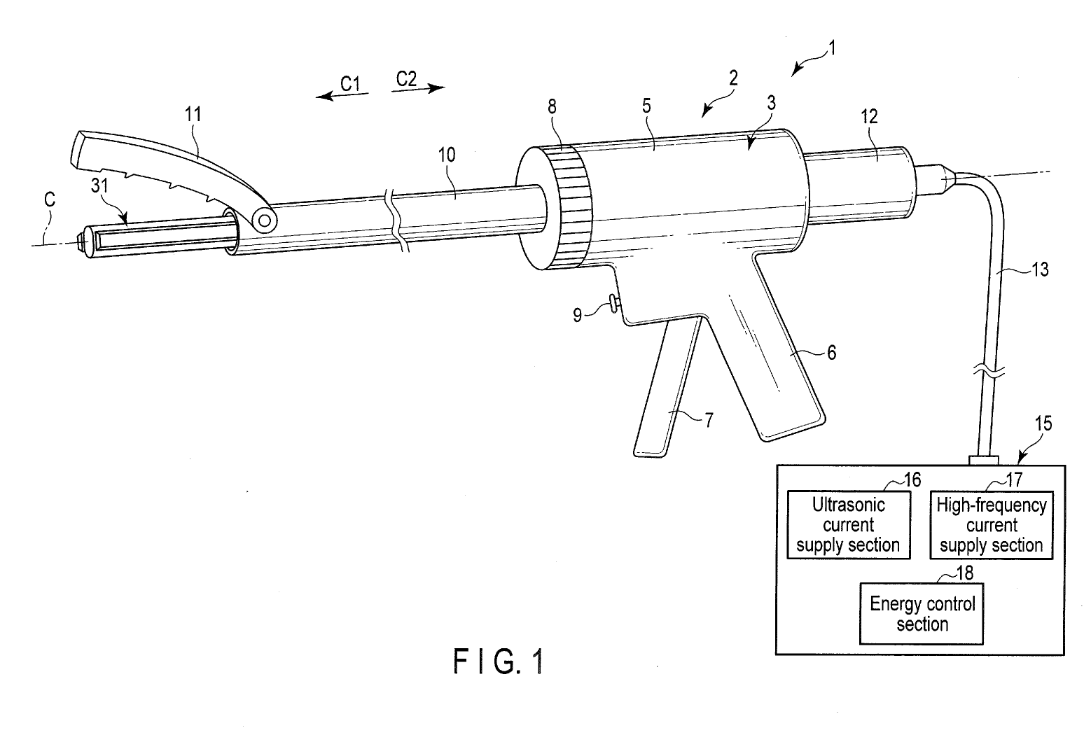

A first embodiment of the present invention is described with reference to FIG. 1 to FIG. 6.

FIG. 1 is a diagram showing a configuration of an ultrasonic treatment device 1 according to the present embodiment. As shown in FIG. 1, the ultrasonic treatment device 1 includes a handpiece 2 which is an ultrasonic treatment instrument. The handpiece 2 has a longitudinal axis C. Here, one of directions parallel to the longitudinal axis C is a distal direction (direction of an arrow C1 in FIG. 1), and a direction opposite to the distal direction is a proximal direction (direction of an arrow C2 in FIG. 1). The distal direction and the proximal direction are axially parallel directions. The handpiece 2 is an ultrasonic coagulation-and-cutting treatment instrument configured to coagulate and cut, for example, a living tissue by using ultrasonic vibration.

The handpiece 2 includes a holding unit 3. The holding unit 3 includes a cylindrical case portion 5 extending along the longitudinal axis C, a fixed handle 6 which is formed integrally with the cylindrical case portion 5, and a movable handle 7 which is turnably attached to the cylindrical case portion 5. The movable handle 7 pivots around a position where the movable handle 7 is attached to the cylindrical case portion 5, so that the movable handle 7 performs opening or closing movement relative to the fixed handle 6. The holding unit 3 also includes a rotational operation knob 8 attached to the distal direction side of the cylindrical case portion 5. The rotational operation knob 8 is rotatable around the longitudinal axis C relative to the cylindrical case portion 5. An energy operation input button 9 which is an energy operation input section is provided to the fixed handle 6.

The handpiece 2 includes a sheath 10 extending along the longitudinal axis C. When the sheath 10 is inserted into an inside of the rotational operation knob 8 and an inside of the cylindrical case portion 5 from the distal direction side, the sheath 10 is attached to the holding unit 3. A jaw 11 is pivotably attached to a distal portion of the sheath 10. The movable handle 7 is connected to a movable cylindrical portion (not shown) of the sheath 10 inside the cylindrical case portion 5. A distal end of the movable cylindrical portion is connected to the jaw 11. If the movable handle 7 is opened or closed relative to the fixed handle 6, the movable cylindrical portion moves along the longitudinal axis C. As a result, the jaw 11 turns around a position where the jaw 11 is attached to the sheath 10. The sheath 10 and the jaw 11 are rotatable about the longitudinal axis C relative to the cylindrical case portion 5 together with the rotational operation knob 8.

The handpiece 2 also includes a vibrator case 12 extending along the longitudinal axis C. When the vibrator case 12 is inserted into the inside of the cylindrical case portion 5 from the proximal direction side, the oscillator case 12 is attached to the holding unit 3. Inside the cylindrical case portion 5, the vibrator case 12 is coupled to the sheath 10. The transducer case 12 is rotatable around the longitudinal axis C relative to the cylindrical case portion 5 together with the rotational operation knob 8. One end of a cable 13 is connected to the vibrator case 12. The other end of the cable 13 is connected to a control unit 15. The control unit 15 includes an ultrasonic current supply section 16, a high-frequency current supply section 17, and an energy control section 18. Here, the control unit 15 is an energy generator including, for example, a central processing unit (CPU) or an application specific integrated circuit (ASIC). The ultrasonic current supply section 16 and the high-frequency current supply section 17 are, for example, electricity sources provided in the energy generator. The energy control section 18 is formed by, for example, electronic circuits (control circuits) provided in the CPU or the ASIC.

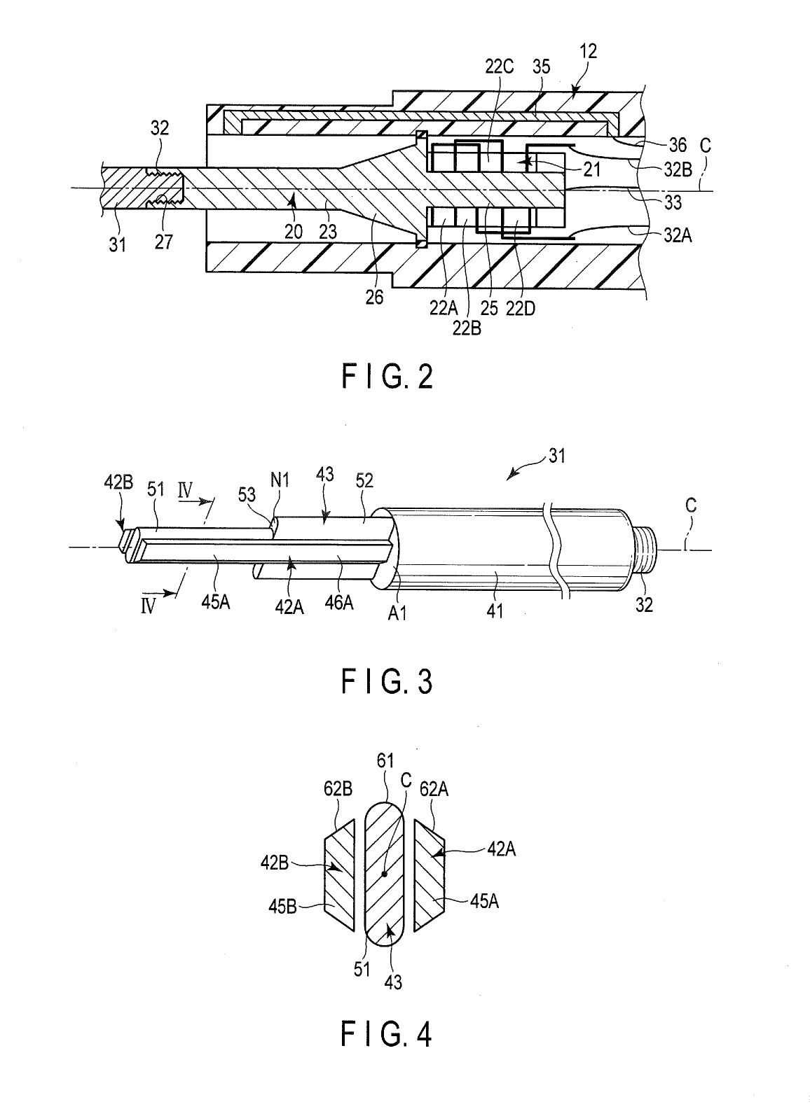

FIG. 2 is a diagram showing an internal configuration of the vibrator case 12. As shown in FIG. 2, the handpiece 2 includes a vibrating body unit 20. The vibrating body unit 20 extends along the longitudinal axis C from within the vibrator case 12 through the inside of the cylindrical case portion 5 and an inside of the sheath 10. The vibrating body unit 20 is rotatable around the longitudinal axis C relative to the cylindrical case portion 5 together with the rotational operation knob 8.

The vibrating body unit 20 includes an ultrasonic vibrator 21 which is an ultrasonic generator configured to generate an ultrasonic vibration when supplied with an electric current. The ultrasonic vibrator 21 includes (four, in the present embodiment) piezoelectric elements 22A to 22D which are configured to convert the electric current to the vibration. The ultrasonic oscillator 21 is disposed inside the vibrator case 12.

The vibrating body unit 20 also includes a columnar member 23 extending along the longitudinal axis C. The columnar member 23 includes a vibrator attachment portion 25. Members such as the piezoelectric elements 22A to 22D that form the ultrasonic oscillator 21 are attached to the vibrator attachment portion 25. A horn portion 26 is formed in the columnar member 23. The sectional area of the horn portion 26 perpendicular to the longitudinal axis C decreases toward the distal direction. One node position of the ultrasonic vibration is located in the horn portion 26. Thus, the amplitude of the ultrasonic vibration is increased in the horn portion 26. An internal thread 27 is provided in a distal portion of the columnar member 23.

The vibrating body unit 20 includes an ultrasonic probe 31 extending along the longitudinal axis C in a part located on the distal direction side with respect to the columnar member 23. An external thread 32 is provided in a proximal portion of the ultrasonic probe 31. When the external thread 32 is screwed into the internal thread 27, the ultrasonic probe 31 is connected to the distal direction side of the columnar member 23. The columnar member 23 extends up to the inside of the cylindrical case portion 5, and the ultrasonic probe 31 is connected to the columnar member 23 inside the cylindrical case portion 5. The ultrasonic probe 31 extends through the inside of the sheath 10, and projects toward the distal direction from a distal end of the sheath 10.

One end of each of electric wiring lines 32A and 32B is connected to the ultrasonic transducer 21. Each of the electric wiring lines 32A and 32B has the other end connected to the ultrasonic current supply section 16 of the control unit 15 through an inside of the cable 13. The ultrasonic vibration is generated in the ultrasonic vibrator 21 by the supply of an ultrasonic generating current to the ultrasonic vibrator 21 from the ultrasonic current supply section 16 via the electric wiring lines 32A and 32B. The generated ultrasonic vibration is then transmitted in the vibrating body unit 20 from the proximal direction toward the distal direction. Accordingly, the ultrasonic vibration is transmitted to the ultrasonic probe 31 from the ultrasonic vibrator 21 via the columnar member 23. The ultrasonic vibration is then transmitted along the longitudinal axis C from the proximal direction to the distal direction in the ultrasonic probe 31.

Regarding the ultrasonic vibration of the vibrating body unit 20, a proximal end of the vibrating body unit 20 (a proximal end of the ultrasonic vibrator 21) and a distal end of the vibrating body unit 20 (a distal end of the ultrasonic probe 31) serve as the anti-node positions of the ultrasonic vibration. Therefore, the vibrating body unit 20 vibrates at a predetermined frequency f0 at which the proximal end of the vibrating body unit 20 and the distal end of the vibrating body unit 20 serve as the loop positions of the ultrasonic vibration. The ultrasonic vibration is a longitudinal vibration having a vibrating direction and a vibration transmission direction that are parallel to the longitudinal axis C.

One end of an electric wiring line 33 is connected to the columnar member 23. The other end of the electric wiring line 33 is connected to the high-frequency current supply section 17 of the control unit 15 through the inside of the cable 13. Thus, a probe-side electric current path of the high-frequency current supplied from the high-frequency current supply section 17 is formed from the high-frequency current supply section 17 to the ultrasonic probe 31 through the electric wiring line 33 and the columnar member 23.

An electric conducting portion 35 is formed in the vibrator case 12. One end of an electric wiring line 36 is connected to the electric conducting portion 35. The other end of the electric wiring line 36 is connected to the high-frequency current supply section 17 of the control unit 15 through the inside of the cable 13. When the vibrator case 12 is coupled to the sheath 10, the sheath 10 is electrically connected to the electric conducting portion 35 of the vibrator case 12. As a result, a jaw-side current path of the high-frequency current supplied from the high-frequency current supply section 17 is formed from the high-frequency current supply section 17 to the jaw 11 through the electric wiring line 36, the electric conducting portion 35 of the vibrator case 12, and the sheath 10.

The energy control section 18 is configured to control L5 the supply state of the ultrasonic generating current from the ultrasonic current supply section 16 and the supply state of the high-frequency current from the high-frequency current supply section 17 in accordance with the input of an energy operation in the energy operation input button 9. A switch (not shown) is provided inside the fixed handle 6. When the energy operation input button 9 is pressed and an energy operation is input, the switch is turned on. The switch is electrically connected to the energy control section 18. When the switch is turned on, an electric signal is transmitted to the energy control section 18, and the input of the energy operation is detected. In response to the detection of the input of the energy operation, the ultrasonic generating current is supplied from the ultrasonic current supply section 16, and the high-frequency current is supplied from the high-frequency current supply section 17.

FIG. 3 is a diagram showing a configuration of the ultrasonic probe 31. As shown in FIG. 3, the ultrasonic probe includes a columnar probe body 41. When the ultrasonic probe 31 is attached to the columnar member 23, the probe body 41 is continuous to the distal direction side of the columnar member 23. On the distal direction side with respect to the probe body 41, first vibrating bodies 42A and 42B and a second vibrating body 43 extend along the longitudinal axis C. That is, the probe body 41 extends along the longitudinal axis C in a part located to the proximal direction side with respect to the first vibrating bodies 42A and 42B and the second vibrating body 43.

A distal end of the probe body 41 is located at one anti-node position A1 of the ultrasonic vibration. Proximal ends of the first vibrating bodies 42A and 42B and a proximal end of the second vibrating body 43 are also located at the loop position A1. Therefore, at one anti-node position A1 of the ultrasonic vibration, the proximal ends of the first vibrating bodies 42A and 42B and the proximal end of the second vibrating body 43 are continuous with the distal end of the probe body 41. The second vibrating body 43 extends in the same range as the first vibrating bodies 42A and 42B in the axially parallel directions parallel with the longitudinal axis C. The first vibrating bodies 42A and 42B extend in the same range with respect to each other in the axially parallel directions. In the present embodiment, the distal end of the ultrasonic probe 31 (the distal end of the vibrating body unit 20) is formed by distal ends of the first vibrating bodies 42A and 42B and a distal end of the second vibrating body 43.

FIG. 4 is a sectional view taken along the line IV-IV in FIG. 3. As shown in FIG. 3 and FIG. 4, each of the first vibrating bodies 42A and 42B and the second vibrating body 43 are discontinuous with respect to each other over the entire length in the axially parallel directions parallel to the longitudinal axis C. The first vibrating bodies 42A and 42B are discontinuous with respect to each other over the entire length in the axially parallel directions. Therefore, in the ultrasonic vibration of the ultrasonic probe 31, one vibrating body (the probe body 41) vibrates in a part located on the proximal direction side from the anti-node position A1, and a plurality of vibrating bodies (three vibrating bodies in the present embodiment) (the first vibrating bodies 42A and 42B and the second vibrating bodies 43) vibrate in a part located on the distal direction side from the loop position A1. However, the vibrating body unit 20 vibrates at the predetermined frequency f0, so that the probe body 41, the first vibrating bodies 42A and 42B, and the second vibrating body 43 vibrate at the same predetermined frequency f0 in the ultrasonic vibration of the ultrasonic probe 31.

Each of the first vibrating bodies 42A and 42B includes a first distal treatment section 45A or 45B at a distal portion thereof. The first distal treatment sections 45A and 45B are provided in the same range with respect to each other in the axially parallel directions. A distal end of the first distal treatment section 45A serves as the distal end of the first vibrating body 42A, and a distal end of the first distal treatment section 45B serves as the distal end of the first vibrating body 42B. In each of the first vibrating bodies 42A and 42B, a first intermediary transmission portion 46A or 46B is provided to the proximal direction side with respect to the first distal treatment section 45A or 45B. The first intermediary transmission portions 46A and 46B are provided in the same range with respect to each other in the axially parallel directions. The ultrasonic vibration is transmitted to the first intermediary transmission portion 46A of the first vibrating body 42A from the distal end of the probe body 41, and transmitted to the first distal treatment section 45A. The ultrasonic vibration is also transmitted to the first intermediary transmission portion 46B of the first vibrating body 42B from the distal end of the probe body 41, and transmitted to the first distal treatment section 45B.

The second vibrating body 43 includes a second distal treatment section 51 at a distal portion thereof. A distal end of the second distal treatment section 51 serves as the distal end of the second vibrating body 43. The second distal treatment section 51 is provided in the same range as the first distal treatment sections 45A and 45B in the axially parallel directions. In the second vibrating body 43, a second intermediary transmission portion 52 is provided to the proximal direction side with respect to the second distal treatment section 51. The second intermediary transmission portion 52 is provided in the same range as the first intermediary transmission portions 46A and 46B in the axially parallel directions. The ultrasonic vibration is transmitted to the second intermediary transmission portion 52 of the second vibrating body 43 from the distal end of the probe body 41, and transmitted to the second distal treatment section 51.

Since each of the first vibrating bodies 42A and 42B and the second vibrating body 43 are discontinuous with respect to each other, the ultrasonic vibration is not transmitted between each of the first vibrating bodies 42A and 42B and the second vibrating body 43. The ultrasonic vibration is not transmitted between the first vibrating body 42A and the first vibrating body 42B either. When the high-frequency current is supplied from the high-frequency current supply section 17, the first distal treatment sections 45A and 45B and the second distal treatment section 51 have a first electric potential E1 in the probe-side electric current path. Thus, when the high-frequency current is supplied from the high-frequency current supply section 17, the first distal treatment sections 45A and 45B and the second distal treatment section 51 function as probe electrode portions.

In the ultrasonic vibration, the probe body 41 vibrates with a first amplitude V1. Since the ultrasonic vibration is transmitted at the anti-node position A1 between the probe body 41 and each of the first vibrating bodies 42A and 42B, the amplitude of the ultrasonic vibration is not increased from the first amplitude V1. The sectional area of each of the first vibrating bodies 42A and 42B perpendicular to the longitudinal axis C does not change over the entire length in the axially parallel directions. Thus, the amplitude of the ultrasonic vibration does not change from the first amplitude V1 in each of the first vibrating bodies 42A and 42B. Therefore, the first intermediary transmission portions 46A and 46B and the first distal treatment sections 45A and 45B vibrate at the predetermined frequency f0 and with the first amplitude V1 when the ultrasonic vibration is transmitted.

Since the ultrasonic vibration is transmitted at the loop position A1 between the probe body 41 and the second vibrating body 43, the amplitude of the ultrasonic vibration is not increased from the first amplitude V1. The sectional area of the second intermediary transmission portion 52 of the second vibrating body 43 perpendicular to the longitudinal axis C does not change over the entire length in the axially parallel directions. Thus, the amplitude of the ultrasonic vibration does not change from the first amplitude V1 in the second intermediary transmission portion 52. Therefore, the second intermediary transmission portion 52 vibrates at the predetermined frequency f0 and with the first amplitude V1 when the ultrasonic vibration is transmitted.

Here, the second vibrating body 43 is provided with a sectional area changing portion 53 in which the sectional area of the second vibrating body 43 perpendicular to the longitudinal axis C changes. The sectional area changing portion 53 is located at one node position N1 of the ultrasonic vibration between the second distal treatment section 51 and the second intermediary transmission portion 52. That is, the sectional area changing portion 53 is provided at one node position N1 of the ultrasonic vibration located to the proximal direction side with respect to the second distal treatment section 51. In the second distal treatment section 51 located to the distal direction side of the sectional area changing portion 53, the sectional area of the second vibrating body 43 perpendicular to the longitudinal axis C is smaller than in the second intermediary transmission portion 52 located to the proximal direction side of the sectional area changing portion 53. Therefore, because of the sectional area changing portion 53, the sectional area of the second vibrating body 43 perpendicular to the longitudinal axis C in a part located to the distal direction side (the transmission direction side of the ultrasonic vibration) from the node position N1 is smaller than the sectional area of the second vibrating body 43 perpendicular to the longitudinal axis C in a part located to the proximal direction side from the node position N1.

The sectional area of the second vibrating body 43 perpendicular to the longitudinal axis C decreases at the node position N1 of the ultrasonic vibration, so that the amplitude of the ultrasonic vibration increases in the sectional area changing portion 53. In the sectional area changing portion 53, the amplitude of the ultrasonic vibration is increased to a second amplitude V2 greater than the first amplitude V1 from the first amplitude V1. Therefore, the second distal treatment portion 51 located to the distal direction side of the sectional area changing portion 53 vibrates with the second amplitude V2 greater than the first amplitude V1 when the ultrasonic vibration is transmitted. However, the second distal treatment section 51 also vibrates at the same predetermined frequency f0 as the first distal treatment sections 45A and 45B.

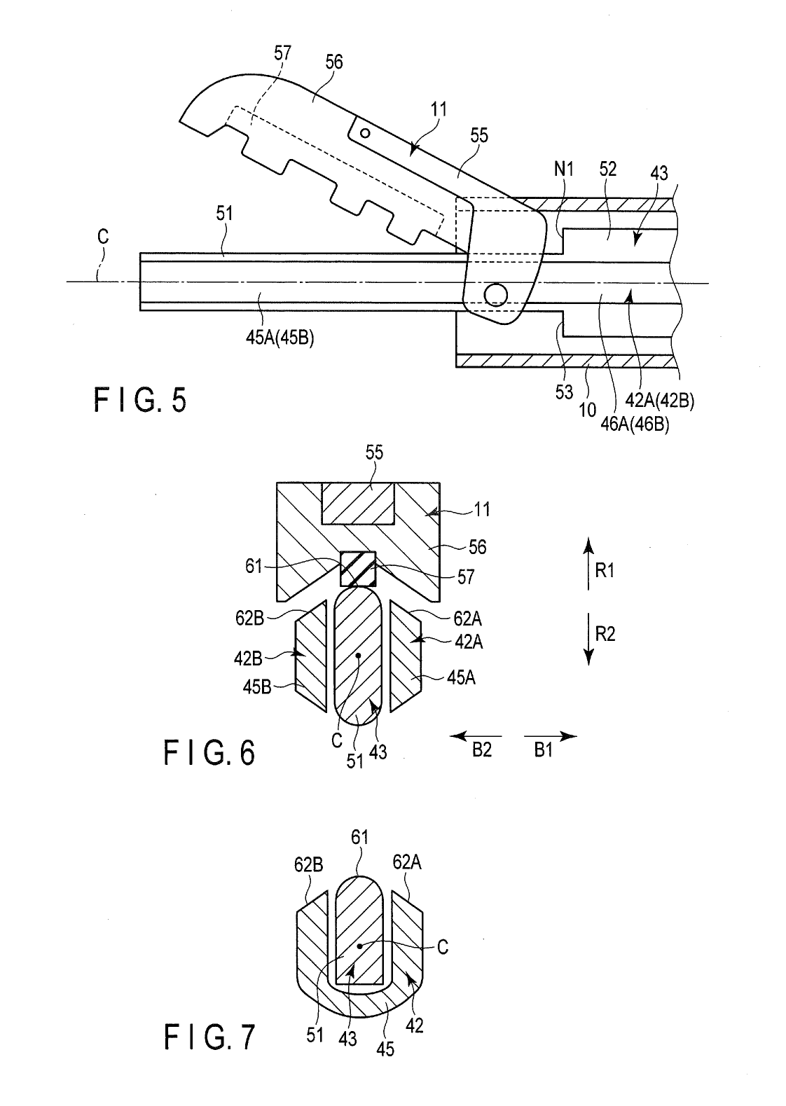

FIG. 5 is a diagram showing a configuration of a distal portion of the handpiece 2. As shown in FIG. 5, the ultrasonic probe 31 is inserted through the sheath 10 so that the first distal treatment sections 45A and 45B and the second distal treatment section 51 project toward the distal direction. Thus, when the jaw 11 pivots relative to the sheath 10, the jaw 11 opens or closes relative to the first distal treatment sections 45A and 45B and the second distal treatment section 51 (opening or closing movement is performed). The sectional area changing portion 53 of the second vibrating body 43 is located inside the sheath 10. That is, the sectional area changing portion 53 of the second vibrating body 43 is located to the proximal direction side with respect to the distal end of the sheath 10.

FIG. 6 is a diagram showing the jaw 11 and the distal portion of the ultrasonic probe 31 in a section perpendicular to the longitudinal axis C. In FIG. 6, the jaw 11 is closed relative to the first distal treatment sections 45A and 45B and the second distal treatment section 51. As shown in FIG. 5 and FIG. 6, the jaw 11 includes a jaw body 55 which is attached to the sheath 10, and a jaw electrode portion 56 which is attached to the jaw body 55. The jaw body 55 and the jaw electrode portion 56 are made of a conducting material. The jaw body 55 and the jaw electrode portion 56 constitute part of the jaw-side current path. When the high-frequency current is supplied from the high-frequency current supply section 17 through the jaw-side current path, the jaw electrode portion 56 has a second electric potential E2 different from the first electric potential E1. A jaw-side abutment portion 57 is attached to the jaw electrode portion 56. The jaw-side abutment portion 57 is made of an insulating material.

An abutment surface 61 which faces the jaw 11 is provided on the second distal treatment section 51. If the jaw 11 is closed relative to the first distal treatment sections 45A and 45B and the second distal treatment section 51 while there is not a treatment target (grasping target) such as a living tissue between the jaw 11 and the first distal treatment sections 45A and 45B as well as the second distal treatment section 51, the jaw-side abutment portion 57 abuts on the abutment surface 61 of the second distal treatment section 51. That is, while the jaw 11 is closed relative to the first distal treatment sections 45A and 45B and the second distal treatment section 51, the jaw-side abutment portion 57 of the jaw 11 can abut on the abutment surface 61 of the second distal treatment section 51.

Noncontact surface 62A or 62B which faces the jaw electrode portion 56 of the jaw 11 are provided to each of the first distal treatment sections 45A and 45B. Here, one of directions perpendicular to the longitudinal axis C and perpendicular to the open-and-close directions (directions of an arrow R1 and an arrow R2 in FIG. 6) of the jaw 11 is a first width direction (direction of an arrow B1 in FIG. 6), and a direction opposite to the first width direction is a second width direction (direction of an arrow B2 in FIG. 6). The noncontact surface 62A is located on the first width direction side of the abutment surface 61, and the noncontact surface 62A is located on the second width direction side of the abutment surface 61. While the jaw-side abutment portion 57 of the jaw 11 is in abutment with the abutment surface 61 of the second distal treatment section 51, the jaw electrode portion 56 has a gap between the jaw electrode portion 56 and the noncontact surfaces 62A and 62B facing the jaw electrode portion 56. That is, the jaw 11 does not contact the noncontact surfaces 62A and 62B of the first distal treatment portions 45A and 45B.

As described above, in the jaw 11, the jaw-side abutment portion 57 made of the insulating material can contact the second distal treatment section 51, whereas the jaw electrode portion 56 made of the conducting material does not contact the first distal treatment sections 45A and 45B. Therefore, when the treatment target is not grasped between the jaw 11 and the first distal treatment sections 45A and 45B as well as the second distal treatment section 51, the jaw 11 is electrically insulated from the first distal treatment sections 45A and 45B and the second distal treatment section 51. The columnar member 23 and the vibrator case 12 are electrically insulated from each other, and the ultrasonic probe 31 and the sheath 10 are electrically insulated from each other. Therefore, when the treatment target is not grasped between the jaw 11 and the first distal treatment sections 45A and 45B as well as the second distal treatment section 51, the probe-side electric current path of the high-frequency current and the jaw-side current path are electrically insulated from each other.

Now, the functions and advantageous effects of the ultrasonic probe 31 and the ultrasonic treatment device 1 are described. When a treatment target such as a living tissue is treated by the use of the ultrasonic treatment device 1, the movable handle 7 is closed relative to the fixed handle 6 so that the jaw 11 is closed relative to the first distal treatment sections 45A and 45B and the second distal treatment section 51. As a result, the treatment target (grasping target) is grasped between the jaw 11 and the first distal treatment sections 45A and 45B as well as the second distal treatment section 51 (the distal portion of the ultrasonic probe 31). In this state, an energy operation is input by the energy operation input button 9. The energy control section 18 then detects the input of the energy operation, an ultrasonic generating current is supplied from the ultrasonic current supply section 16 to the ultrasonic vibrator 21, and a high-frequency current is supplied from the high-frequency current supply section 17.

When the ultrasonic generating current is supplied, the ultrasonic vibration is generated in the ultrasonic vibrator 21, and the generated ultrasonic vibration is transmitted to the ultrasonic probe 31. In the ultrasonic probe 31, the ultrasonic vibration is transmitted to the first vibrating bodies 42A and 42B from the distal end of the probe body 41, and the ultrasonic vibration is transmitted to the second vibrating body 43 from the distal end of the probe body 41. At the anti-node position A1 of the ultrasonic vibration where the distal end of the probe body 41 (the proximal ends of the first vibrating bodies 42A and 42B and the proximal end of the second vibrating body 43) is located, the number of vibrating body or vibrating bodies changes from a condition in which one vibrating body (41) vibrates to a condition in which the plurality of vibrating bodies (42A and 42B, and 43) vibrate. At the position where the number of vibrating body or vibrating bodies changes, the ultrasonic vibration is easily affected by stress in directions perpendicular to the longitudinal axis C. When the ultrasonic vibration is affected by the stress, the vibration mode of the ultrasonic vibration changes, and the vibrating body unit 20 (ultrasonic probe 31) does not correctly perform the longitudinal vibration. As a result, the ultrasonic vibration is incorrectly transmitted to the first distal treatment sections 45A and 45B and the second distal treatment section 51.

Therefore, in the present embodiment, the number of member which vibrates or members which vibrate is set to change at the loop position A1. At the anti-node positions of the ultrasonic vibration including the anti-node position A1, a displacement resulting from vibration is maximized, but the stress in the directions perpendicular to the longitudinal axis C is zero. Therefore, no stress affects the ultrasonic vibration at the anti-node position A1 where the number of member which vibrates or members which vibrate changes. Thus, the vibration mode is not changed by the change in the number of member which vibrates or members which vibrate, and the vibrating body unit 20 correctly produces the longitudinal vibration. As a result, the ultrasonic vibration can be correctly transmitted to the first distal treatment sections 45A and 45B of the ultrasonic probe 31 and the second distal treatment section 51.

The ultrasonic vibration transmitted to each of the first vibrating bodies 42A and 42B from the probe body 41 is transmitted to each of the first distal treatment sections 45A and 45B so that the amplitude is not increased from the first amplitude V1 in the probe body 41. Thus, when the ultrasonic vibration is transmitted, each of the first distal treatment sections 45A and 45B vibrates at the predetermined frequency f0 and with the same first amplitude V1 as the probe body 41. Therefore, each of the first distal treatment sections 45A and 45B vibrates with the first amplitude V1 which does not increase more than necessary.

The amplitude of the ultrasonic vibration transmitted to the second vibrating body 43 from the probe body 41 is increased in the sectional area changing portion 53 located to the proximal direction side with respect to the second distal treatment section 51. The amplitude of the ultrasonic vibration is increased to the second amplitude V2 from the first amplitude V1 in the sectional area changing portion 53 located at the node position N1. Thus, when the ultrasonic vibration is transmitted, the second distal treatment section 51 vibrates at the predetermined frequency f0 and with the second amplitude V2 greater than the first amplitude V1. Therefore, the second distal treatment section 51 vibrates with sufficiently large second amplitude V2.

When the second vibrating body 43 (the ultrasonic probe 31) vibrates while the treatment target is being grasped between the jaw 11 and the distal portion of the ultrasonic probe 31, a frictional heat is generated between the abutment surface 61 of the second vibrating body 43 and the treatment target. The treatment target is cut by the frictional heat. Since the second vibrating body 43 vibrates with the sufficiently large second amplitude V2, cutting performance is ensured. That is, cutting performance can be ensured in the cutting treatment which is conducted by use of the second distal treatment section 51 and the abutment surface 61.

When the high-frequency current is supplied from the high-frequency current supply section 17, the first distal treatment sections 45A and 45B and the second distal treatment section 51 have the first electric potential E1, and the jaw electrode portion 56 of the jaw 11 has the second electric potential E2 different from the first electric potential E1. When the high-frequency current is supplied while the treatment target is being grasped, the high-frequency current runs through the treatment target between the jaw electrode portion 56 of the jaw 11 and the noncontact surfaces 62A and 62B of the first distal treatment sections 45A and 45B facing the jaw electrode portion 56. As a result, the treatment target is denatured and coagulated. In this case, each of the first vibrating bodies 42A and 42B vibrates with such a degree of the first amplitude V1 that the treatment target is not firmly adhered, and the coagulation performance is therefore ensured. That is, treatment performance can be ensured in the coagulation treatment which is conducted by the use of the noncontact surfaces 62A and 62B of the first distal treatment sections 45A and 45B.

The degree of the first amplitude V1 in the first distal treatment sections 45A and 45B is such that the treatment target is not firmly adhered, and does not increase more than necessary. Thus, in the ultrasonic probe 31, parts other than the second distal treatment section 51 (i.e., the probe body 41, the first vibrating bodies 42A and 42B, and the second intermediary transmission portion 52), the second distal treatment section 51 vibrating with the second amplitude V2, vibrate with the first amplitude V1 which does not increase more than necessary. That is, in the ultrasonic probe 31, parts other than the second distal treatment section 51 vibrate with a degree of the first amplitude V1 sufficient to ensure the treatment performance of the coagulation treatment. Since the amplitude (first amplitude V1) of the ultrasonic vibration does not increase in the parts of the ultrasonic probe 31 other than the second distal treatment section 51, a load of the ultrasonic vibration on the ultrasonic probe 31 does not increase. Since no high load is applied by the ultrasonic vibration, the strength of the elongated ultrasonic probe 31 during vibration can be ensured.

(Modifications)

Although the two first vibrating bodies 42A and 42B are provided in the first embodiment, this is not a limitation. For example, as in a first modification shown in FIG. 7, only one first vibrating body 42 may be provided. In the present modification, a first distal treatment section 45 is provided at the distal portion of the first vibrating body 42, and the second distal treatment section 51 is provided at the distal portion of the second vibrating body 43. In the present modification as well, the first vibrating body 42 vibrates with the first amplitude V1 which does not increase more than necessary. The second vibrating body 43 is discontinuous with the first vibrating body 42 over the entire length in the axially parallel directions parallel with the longitudinal axis C. In the present modification as well, in the second vibrating body 43, the sectional area changing portion 53 is provided at one node position N1 of the ultrasonic vibration located to the proximal direction side with respect to the second distal treatment section 51. In the second vibrating body 43, the amplitude of the ultrasonic vibration is increased to the second amplitude V2 from the first amplitude V1 in the sectional area changing portion 53, and then the ultrasonic vibration is transmitted to the second distal treatment section 51. Therefore, the second distal treatment section 51 vibrates with the sufficiently large second amplitude V2.

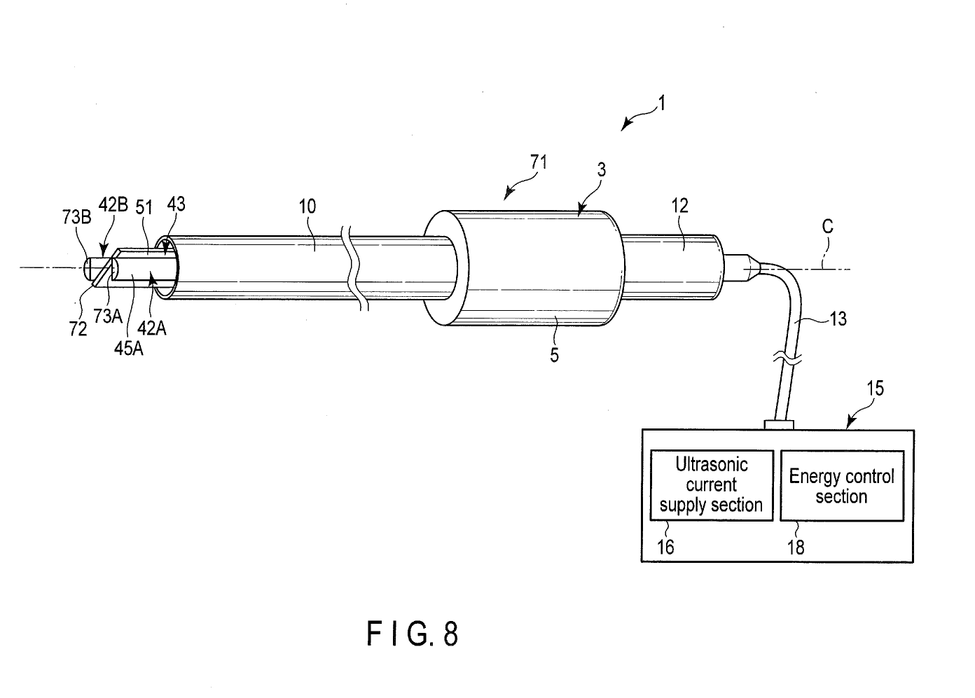

Although the handpiece 2 according to the first embodiment is a grasping treatment instrument configured to grasp and treat a treatment target between the distal portion of the ultrasonic probe 31 and the jaw 11, this is not a limitation. For example, as in a second modification shown in FIG. 8, an ultrasonic treatment instrument 71 may be used instead of the handpiece 2. The vibrator case 12, the holding unit 3, the sheath 10, and the vibrating body unit 20 are provided in the ultrasonic treatment instrument 71, as in the handpiece 2. However, in the ultrasonic treatment instrument 71, the holding unit 3 only includes the cylindrical case portion 5, and is not provided with the fixed handle 6, the movable handle 7, and the rotational operation knob 8. The vibrating body unit 20 includes the ultrasonic probe 31, the columnar member 23, and the ultrasonic vibrator 21, but the ultrasonic treatment instrument 71 is not provided with the jaw 11. In the present modification, the high-frequency current supply section 17 is not provided in the control unit 15, and the high-frequency current is not supplied to the ultrasonic probe 31.

In the present modification as well, the ultrasonic probe 31 includes the probe body 41, the first vibrating bodies 42A and 42B, and the second vibrating body 43. The first distal treatment sections 45A and 45B of the first vibrating bodies 42A and 42B and the second distal treatment section 51 of the second vibrating body 43 project toward the distal direction from the distal end of the sheath 10. In the present modification as well, the first distal treatment sections 45A and 45B vibrate with the first amplitude V1. The second vibrating body 43 is provided with the sectional area changing portion 53, and the sectional area changing portion 53 increases the amplitude of the ultrasonic vibration to the second amplitude V2. Thus, the second distal treatment section 51 vibrates with the second amplitude V2 greater than the first amplitude V1.

A scalpel portion 72 which is sharpened toward the distal direction is provided at the distal end of the second distal treatment section 51. When the second vibrating body 43 vibrates while the scalpel portion 72 is in contact with the treatment target, the treatment target is dissected. In this case, the second vibrating body 43 vibrates with the sufficiently large second amplitude V2, so that the treatment performance in the cutting treatment is ensured.

A coagulation surface 73A or 73B is provided at the distal end of each of the first distal treatment sections 45A and 45B. When the first vibrating bodies 42A and 42B vibrate while the coagulation surfaces 73A and 73B are in contact with the treatment target, the treatment target is coagulated. In this case, the degree of the first amplitude V1 of the first vibrating bodies, 42A and 428 has only to be such that the treatment performance of the coagulation treatment is ensured, and does not increase more than necessary. Thus, the load of the ultrasonic vibration on the ultrasonic probe 31 does not increase, and the strength of the ultrasonic probe 31 during vibration is ensured.

Consequently, an ultrasonic probe (31) has only to include a first vibrating body (42A, 42B; 42) extending along a longitudinal axis (C), and a second vibrating body (43) which extends along the longitudinal axis C in the same range as the first vibrating body (42A, 42B; 42) in axially parallel directions parallel with the longitudinal axis (C) and which is discontinuous with the first vibrating body (42A, 42B; 42) over the entire length in the axially parallel directions. The first vibrating body (42A, 42B; 42) has only to include, at a distal portion thereof, a first distal treatment section (45A, 45B; 45) which vibrates at a predetermined frequency (f0) and with a first amplitude (V1) when the ultrasonic vibration is transmitted. The second vibrating body (43) has only to include, at the distal portion thereof, a second distal treatment section (51) which vibrates at the same predetermined frequency (f0) as the first distal treatment section (45A, 45B; 45) and with a second amplitude (V2) greater than the first amplitude (V1) when the ultrasonic vibration is transmitted.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic probe configured to transmit an ultrasonic vibration from a proximal direction toward a distal direction along a longitudinal axis, the ultrasonic probe comprising:
a first vibrating body which extends along the longitudinal axis, the first vibrating body including, at a distal portion thereof, a first distal treatment section which is configured to vibrate at a predetermined frequency and with a first amplitude when the ultrasonic vibration is transmitted; and
a second vibrating body which extends along the longitudinal axis in a same range as the first vibrating body in axially parallel directions parallel to the longitudinal axis and which is discontinuous with the first vibrating body over an entire length in the axially parallel directions, the second vibrating body including, at a distal portion thereof, a second distal treatment section which is configured to vibrate at the same predetermined frequency as the first distal treatment section and with a second amplitude greater than the first amplitude when the ultrasonic vibration is transmitted.

2. The ultrasonic probe according to claim 1, wherein the second vibrating body includes a sectional area changing portion which is provided at one node position of the ultrasonic vibration located to a proximal direction side with respect to the second distal treatment section and which changes in a sectional area perpendicular to the longitudinal axis of the second vibrating body, the sectional area changing portion being configured to increase an amplitude of the ultrasonic vibration transmitted toward the distal direction in the second vibrating body.

3. An ultrasonic treatment device comprising:
the ultrasonic probe according to claim 2;
an ultrasonic vibrator which is configured to generate the ultrasonic vibration and which is configured to transmit the ultrasonic vibration to the ultrasonic probe; and
a sheath through which the ultrasonic probe is inserted so that the first distal treatment section and the second distal treatment section project toward the distal direction, the sectional area changing portion being located to the proximal direction side with respect to a distal end of the sheath.

4. The ultrasonic probe according to claim 1, further comprising a probe body extending along the longitudinal axis in a part located to a proximal direction side with respect to the first vibrating body and the second vibrating body, a proximal end of the first vibrating body and a proximal end of the second vibrating body being continuous with a distal end of the probe body at one anti-node position of the ultrasonic vibration, the ultrasonic vibration being configured to be transmitted to the first vibrating body and the second vibrating body from the distal end of the probe body.

5. An ultrasonic treatment device comprising:
the ultrasonic probe according to claim 1;
an ultrasonic vibrator which is configured to generate the ultrasonic vibration and which is configured to transmit the ultrasonic vibration to the ultrasonic probe;
a sheath through which the ultrasonic probe is inserted so that the first distal treatment section and the second distal treatment section project toward the distal direction; and
a jaw which is pivotably attached to a distal portion of the sheath and which is configured to pivot relative to the sheath and thereby configured to open or close relative to the first distal treatment section and the second distal treatment section.

6. The ultrasonic treatment device according to claim 5, wherein
the second distal treatment section of the second vibrating body includes an abutment surface which faces the jaw and on which the jaw is abutable when the jaw is closed relative to the first distal treatment section and the second distal treatment section, and
the first distal treatment section of the first vibrating body includes a noncontact surface which faces the jaw and which has a gap between the noncontact surface and the jaw while the jaw is in abutment with the abutment surface.

7. The ultrasonic treatment device according to claim 6, wherein
the jaw includes
a jaw-side abutment portion which is abutable on the abutment surface of the second distal treatment section and which is made of an insulating material, and
a jaw electrode portion which is made of a conducting material and which has a gap between the jaw electrode portion and the noncontact surface of the first distal treatment section facing the jaw electrode portion while the jaw-side abutment portion is in abutment with the abutment surface of the second distal treatment section.
